(19) United States
(12) Patent Application Publication
(10) Pub. No.: US 2017/0058247 A1
(43) Pub. Date: Mar. 2, 2017

(54) ANAEROBIC FERMENTATION WITH VENTURI STIRRING EQUIPMENT

(71) Applicant: Tze-Ming KAO, Taipei (TW)

(72) Inventor: Tze-Ming KAO, Taipei (TW)

(21) Appl. No.: 14/836,581

(22) Filed: Aug. 26, 2015

(51) Int. Cl. C12M 1/00; C12M 1/12

(52) U.S. Cl. CPC C12M 29/06; C12M 23/14; C12M 29/00; C12M 25/06

(57) ABSTRACT

An anaerobic fermentation with venturi stirring equipment has a container, a circulation unit, and a spray pipe. The container has at least one opening. The circulation unit communicates with the container and has a pump, a pumping pipe, a conveying pipe, and at least one storage tank. The pump is disposed out of the container. The pumping pipe communicates with the pump and a top of the container. The conveying pipe communicates with the pump and is mounted through one of the at least one opening, and the conveying pipe is located in the container. A gas valve is mounted on the conveying pipe. The at least one storage tank is mounted on the conveying pipe. The spray pipe is mounted in the container and communicates with the conveying pipe. The anaerobic fermentation with venturi stirring equipment can promote the fermenting rate and the ecological distribution of the anaerobic bacteria.

### BACKGROUND OF THE INVENTION

1. Field of the Invention

The present invention relates to anaerobic fermentation equipment, especially a kind of equipment for anaerobic fermentation treatment of organic substances.

2. Description of the Prior Arts

Biogas is a combustible gas generated from anaerobic fermentation treatment of organic substances. Biogas mainly comprises methane, carbon dioxide and hydrogen sulfide, and the generating source of biogas includes manure, slurry, sewage, garbage and other biodegradable organic substances. The development and the application of biogas are gradually valued because biogas generated from the organic waste materials can be used to generate electricity, used as a fuel for cooking, heating and transportation. The conventional anaerobic fermentation equipment has an air tight container. Organic substances and the culture fluid of anaerobic bacteria are fed into the container and then the container is sealed. So the barrel is isolated from the air. The anaerobic bacteria start reproducing in oxygen absent environment and fermenting the organic substances. However, the conventional fermenting technique requires mechanical stirring in the container to improve digestion of organic substances and sediment, which is difficult to achieve in an air tight container and is energy intensive.

To overcome the shortcomings, the present invention provides an anaerobic fermentation with venturi stirring equipment to mitigate or obviate the aforementioned problems.

### SUMMARY OF THE INVENTION

The main objective of the present invention is to provide an anaerobic fermentation with venturi stirring equipment to promote digestion of organic substances and improve the ecological distribution of the anaerobic bacteria.

The anaerobic fermentation with venturi stirring equipment comprises a container, a circulation unit, and a spray pipe. The container has at least one opening. The circulation unit communicates with the container and has a pump, a pumping pipe, a conveying pipe, and at least one storage tank. The pump is disposed out of the container. The pumping pipe communicates with the pump and a top of the container. The conveying pipe communicates with the pump, is mounted through one of the at least one opening of the container, and has two ends and a gas valve. One of the two ends of the conveying pipe communicates with the pump. The gas valve is mounted on the conveying pipe. The at least one storage tank is mounted on the conveying pipe. The spray pipe is mounted in the container and communicates with the other one of the two ends of the conveying pipe.

The organic substances are fed into the container and bury the spray pipe. Then the opening of the container is sealed and the organic substances start the anaerobic fermentation. Biogas is generated when the organic substances are fermenting. The biogas floats at the top side in the container. After a specific quantity of biogas is accumulated, the pump of the circulation unit starts to draw biogas out of the container by the pumping pipe. The biogas is conveyed from the conveying pipe to the spray pipe, and the biogas is spewed out of the spray pipe to stir and mix the organic substances for promoting digestion of organic substances and improving the ecological distribution of the anaerobic bacteria.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a first embodiment of an anaerobic fermentation with venturi stirring equipment in accordance with the present invention;

FIG. 2 is a front cross-sectional view of the anaerobic fermentation with venturi stirring equipment in FIG. 1, filled with organic substances;

FIG. 3 is a front cross-sectional view of the anaerobic fermentation with venturi stirring equipment in FIG. 1, shown stirring organic substances;

FIG. 4 is a front cross-sectional view of the anaerobic fermentation with venturi stirring equipment in FIG. 1, shown draining the water produced from the organic substances;

FIG. 5 is a front cross-sectional view of a second embodiment of an anaerobic fermentation with venturi stirring equipment in accordance with the present invention, shown filled with organic substances;

FIG. 6 is a front cross-sectional view of the anaerobic fermentation with venturi stirring equipment in FIG. 5, shown stirring the organic substances;

FIG. 7 is a front cross-sectional view of the anaerobic fermentation with venturi stirring equipment in FIG. 5, shown draining the water produced from the organic substances; and

FIG. 8 is a front sectional view of a third embodiment of an anaerobic fermentation with venturi stirring equipment in accordance with the present invention.

### DETAILED DESCRIPTION OF THE PREFERRED EMBODIMENTS

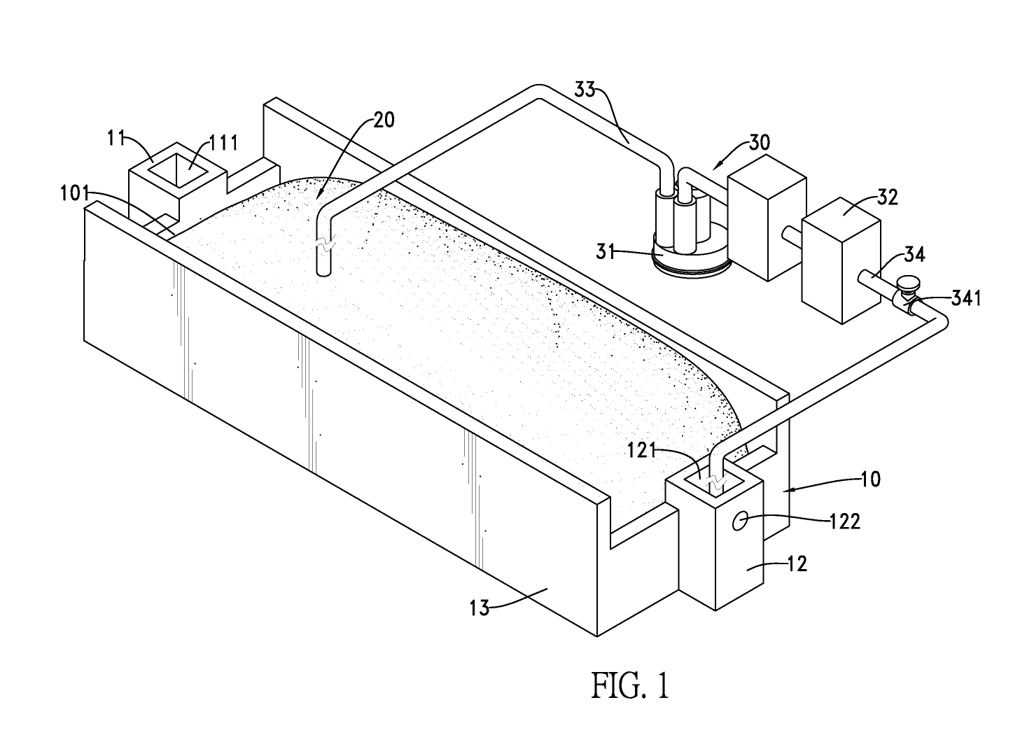

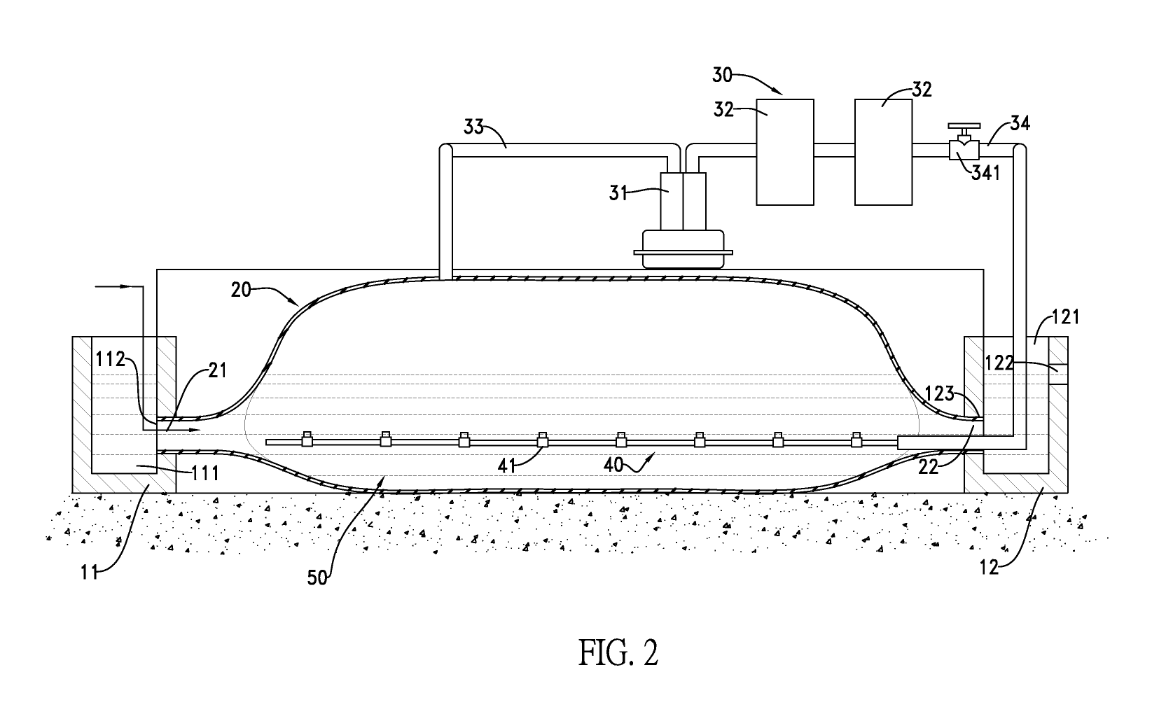

With reference to FIGS. 1 and 2, a first embodiment of an anaerobic fermentation with venturi stirring equipment in accordance with the present invention comprises an enclosure 10, a container 20, a circulation unit 30, and a spray pipe 40.

With reference to FIGS. 1 and 2, the enclosure 10 is a rectangular structure and has a surrounding wall 13, a first transporting base 11 and a second transporting base 12. A space 101 is surrounded by the surrounding wall 13 of the enclosure 10. The first transporting base 11 is formed on the surrounding wall 13 of the enclosure 10 and has a first gateway 111 and a first connecting opening 112. The first gateway 111 is formed in the first transporting base 11. The first connecting opening 112 is formed through the surrounding wall 13 of the enclosure 10 and communicates with the first gateway 111 and the space 101. The second transporting base 12 is mounted on the surrounding wall 13 opposite to the first transporting base 11 and has a second gateway 121, an outlet 122, and a second connecting opening 123. The second gateway 121 is formed in the second transporting base 12. The outlet 122 is formed through the second transporting base 12 and communicates with the second gateway 121. The second connecting opening 123 is formed through the surrounding wall 13 of the enclosure 10 and communicates with the second gateway 121 and the space 101.

With reference to FIGS. 1 and 2, the container 20 is mounted in the space 101 of the enclosure 10 and has a first opening 21 and a second opening 22. The container 20 is a bag. The first opening 21 is formed through the container 20 and faces the first transporting base 11. The first opening 21 communicates with the first gateway 111 of the first transporting base 11 via the first connecting opening 112. The second opening 22 is formed through the container 20 and faces the second transporting base 12. The second opening 22 communicates with the second gateway 121 of the second transporting base 12 via the second connecting opening 123.

The circulation unit 30 communicates with the container 20 and has a pump 31, multiple storage tanks 32, a pumping pipe 33 and a conveying pipe 34. The pump 31 and the multiple storage tanks 32 are disposed out of the enclosure 10. The pumping pipe 33 communicates with the container 20 and the pump 31. The conveying pipe 34 has two ends and a gas valve 341. One of the two ends of the conveying pipe 34 communicates with the pump 31 and the multiple storage tanks 32 are mounted on the conveying pipe 34. The other one of the two ends of the conveying pipe 34 is mounted through the second gateway 121 of the second transporting base 12 and the second opening 22 of the container 20, and is located in the container 20. The gas valve 341 is mounted on the conveying pipe 34 and is distal from the pump 31. The gas valve 341 controls the gas flow in the conveying pipe 34.

With reference to FIGS. 2 to 4, the spray pipe 40 is mounted horizontally in a bottom of the container 20. The spray pipe 40 is connected to the other end of the conveying pipe 34 and has multiple jets 41. The multiple jets 41 are mounted upward on the spray pipe 40. The multiple jets 41 are spaced from each other at intervals.

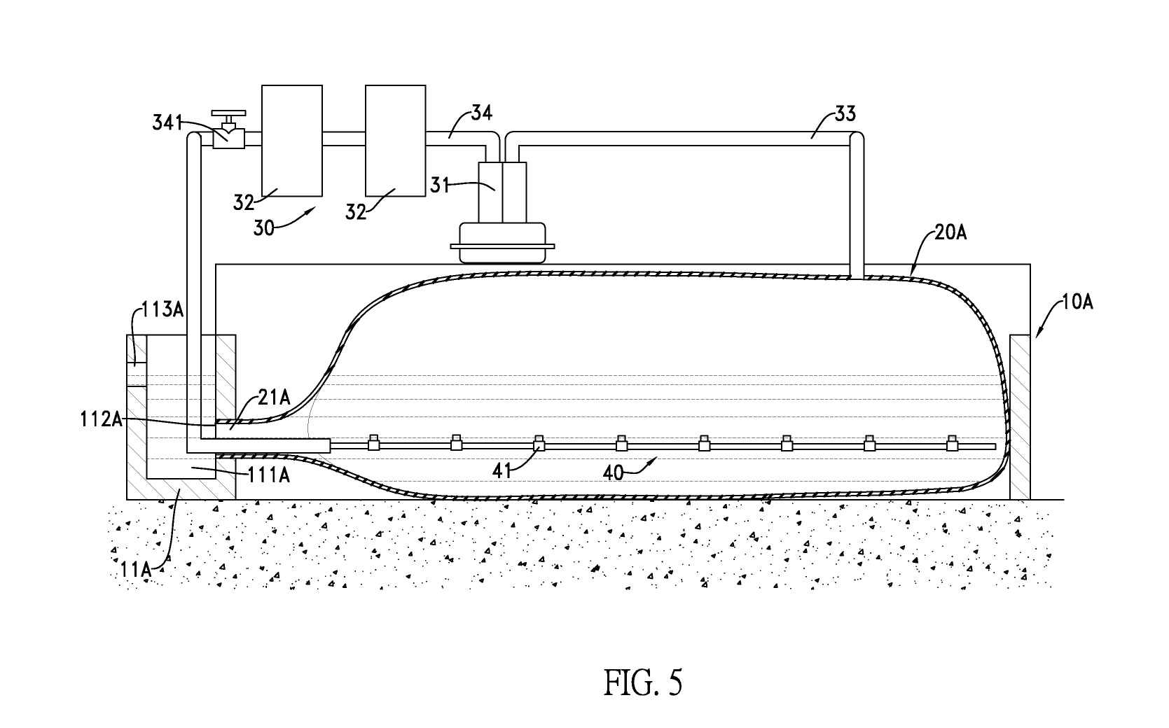

With reference to FIG. 5, a second embodiment of an anaerobic fermentation with venturi stirring equipment in accordance with the present invention comprises an enclosure 10A, a container 20A and a circulation unit 30. The enclosure 10A has a surrounding wall 13, a space 101A, and a transporting base 11A. The space 101A is surrounded by the surrounding wall 13 of the enclosure 10A. The transporting base 11A is mounted on the surrounding wall 13 of the enclosure 10A and has a gateway 111A, a connecting opening 112A, and an outlet 113A. The gateway 111A is formed in the transporting base 11A. The connecting opening 112A is formed through the surrounding wall 13 of the enclosure 10A and communicates with the gateway 111A and the space 101A. The outlet 113A is formed through the transporting base 11A and communicates with the second gateway 111A.

With reference to FIG. 5, the container 20A is a bag structure. The container 20A is mounted in the space 101A of the enclosure 10A and has an opening 21A. The opening 21A is formed through the container 20A and faces the transporting base 11A of the enclosure 10A. The opening 21A communicates with the gateway 111A of the transporting base 11A via the connecting opening 112A. The other one of the two ends of the conveying pipe 34 is mounted through the gateway 111A of the transporting base 11A and the opening 21A of the container 20A, and is located in the container 20A. The conveying pipe 34 of the circulation unit 30 is connected to the spray pipe 40. The circulation unit 30 of the second embodiment is the same as that of the first embodiment.

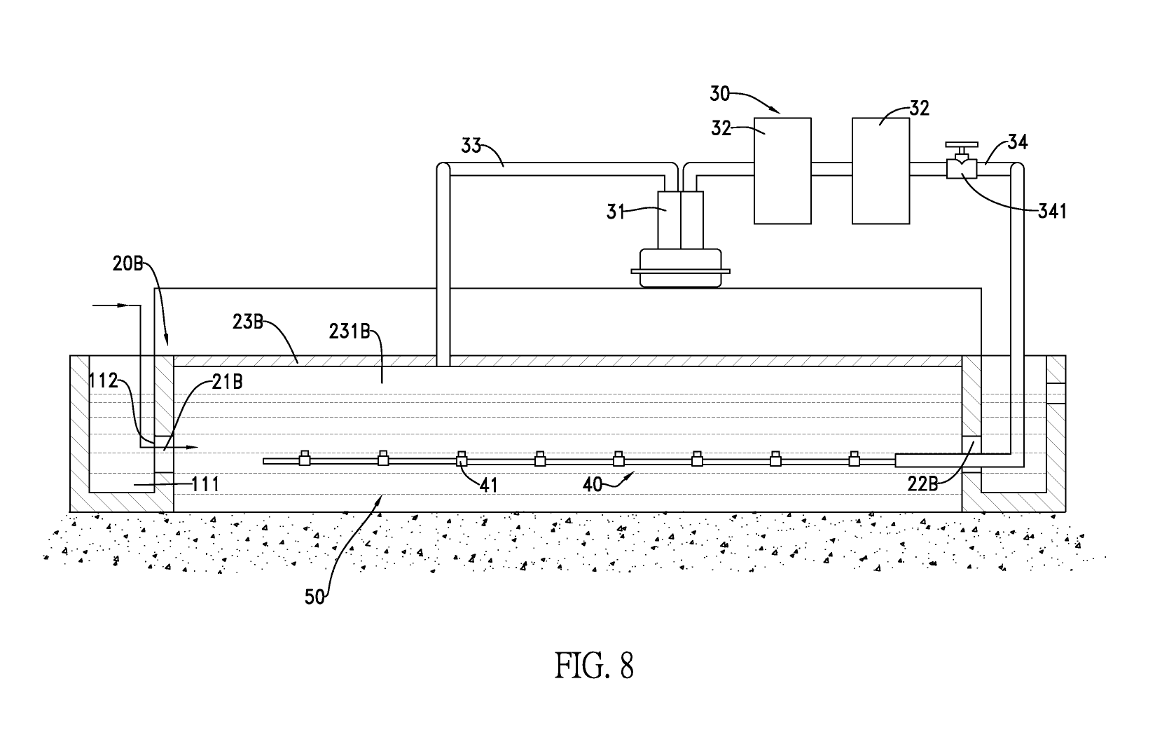

With reference to FIG. 8, a third embodiment of an anaerobic fermentation with venturi stirring equipment in accordance with the present invention is similar to the first embodiment, but the third embodiment does not have an enclosure, and the container 20B is made of cement or bricks and has a surrounding wall 13 having a top opening, a first opening 21B, a second opening 22B, and a cover 23B. The first opening 21B is formed through the surrounding wall 13 of the container 20B. The second opening 22B is formed through the surrounding wall 13 of the container 20B. The cover 23B is hermetically mounted on the top opening of the surrounding wall 13, and an inner space 231B is formed between the surrounding wall 13 and the cover 23B and communicates with the first opening 21B and the second opening 22B. The conveying pipe 34 is located in the inner space 231B of the container 20B and is connected to the spray pipe 40.

With reference to FIGS. 1 to 3 and 8, when the first embodiment and the third embodiment of the present invention are in use, organic substances 50 such as manure, slurry, sewage, leftover are fed into the container 20, 20B via the first gateway 111 of the first transporting base 11 and the first opening 21,21B and bury the spray pipe 40. At the same time, the culture fluids of the anaerobic bacteria are fed into the container 20, 20B via the first gateway 111 and the first opening 21, 21B. The first gateway 111 and the second gateway 121 are sealed by the organic substances 50, so the container 20, 20B is isolated from the air and becomes an anaerobic environment. Then, the anaerobic bacteria start reproducing themselves in the container 20, 20B and fermenting the organic substances 50.

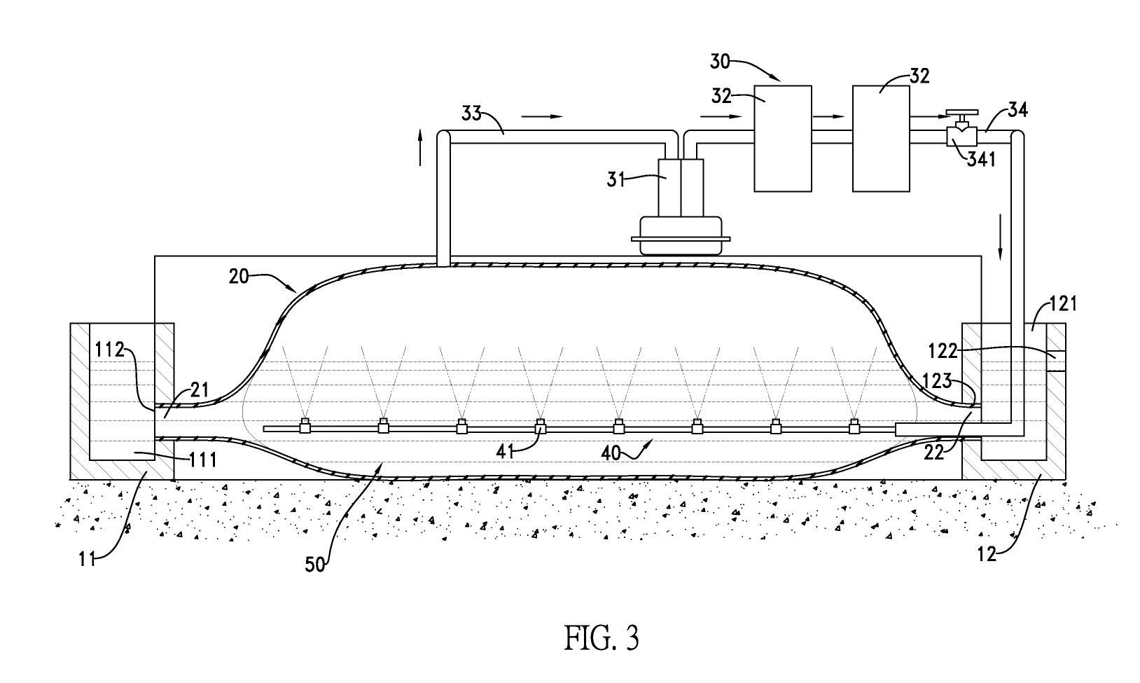

With reference to FIGS. 3 and 8, the organic substances 50 are decomposed to generate biogas, biogas slurry, and organic manure. The biogas is generated when the organic substances are fermenting. The biogas floats at the top side in the container 20, 20B due to the light weight property. The biogas slurry and the organic manure are located at the bottom side of the container 20, 20B. After the biogas is generated, the pump 31 of the circulation unit 30 will start to draw biogas to store in the storage tanks 32 via the pumping pipe 33. And the gas valve 341 is switched on and makes the conveying pipe 34 communicate with the spray pipe 40. So the biogas can be flowed into the spray pipe 40. The biogas is spewed out of the spray pipe 40 at short intervals of time via each one of the jets 41 to stir and mix the organic substances 50 for promoting the rate of anaerobic fermentation and improving the ecological distribution of the anaerobic bacteria.

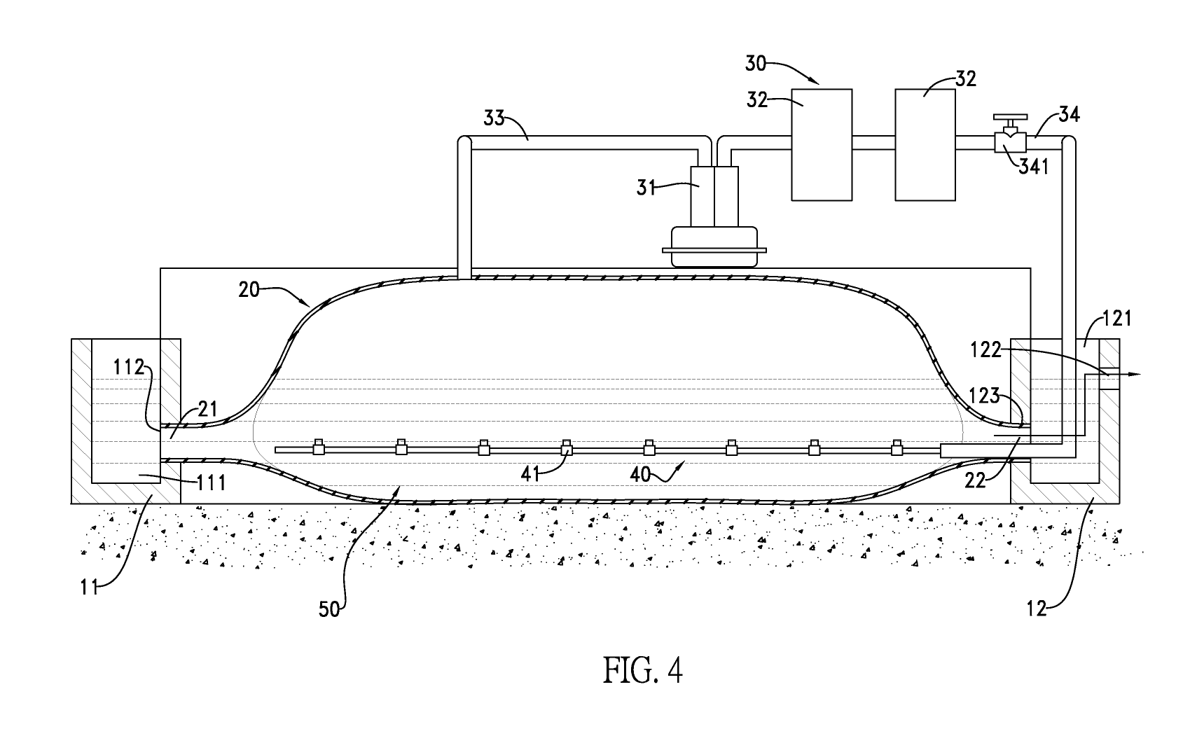

With reference to FIG. 4, after the anaerobic fermentation of the organic substances 50 is completed, all of the biogas will be stored in the multiple storage tanks 32 to generate energy. In the first embodiment, the biogas fluid is discharged from the outlet 122 via the second opening 22 of the container 20 and the second gateway 121 of the second transporting base 12. In the third embodiment, the biogas fluid is discharged from the second opening 22B of the container 20B. The biogas fluid can be applied to build an artificial wetland. The organic manure can be the fertilizer for farming. Thus, the present invention can accomplish the sustainable development of the environment.

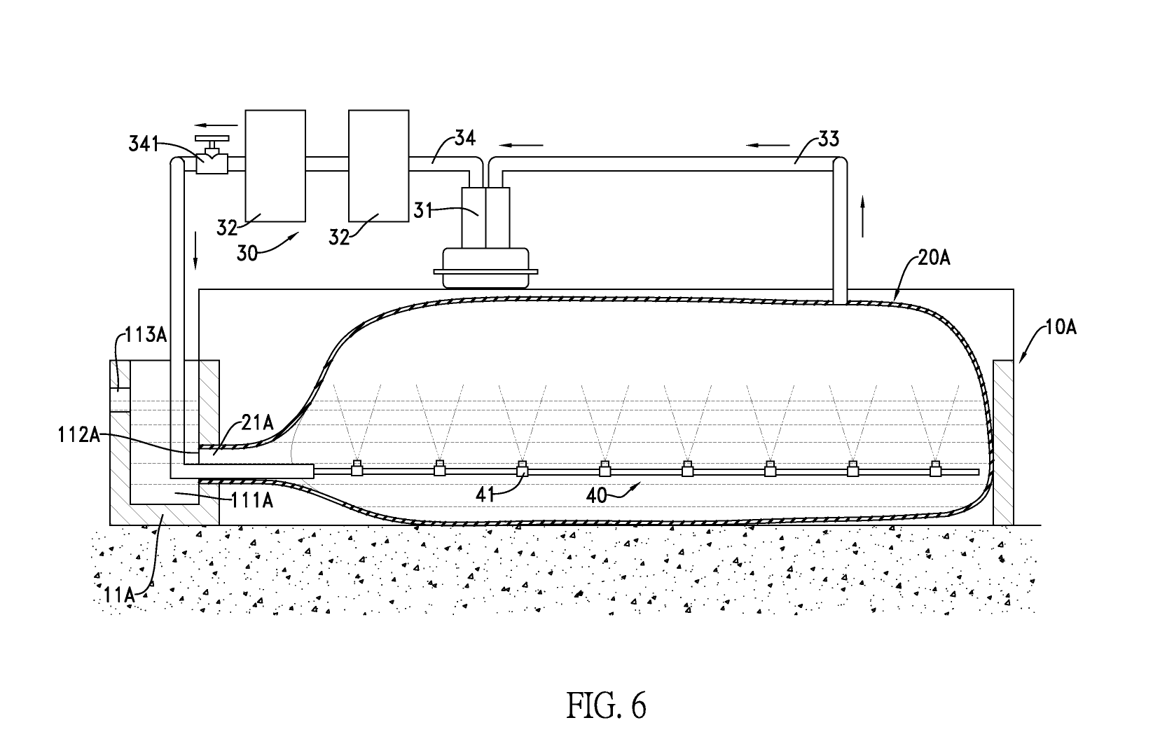

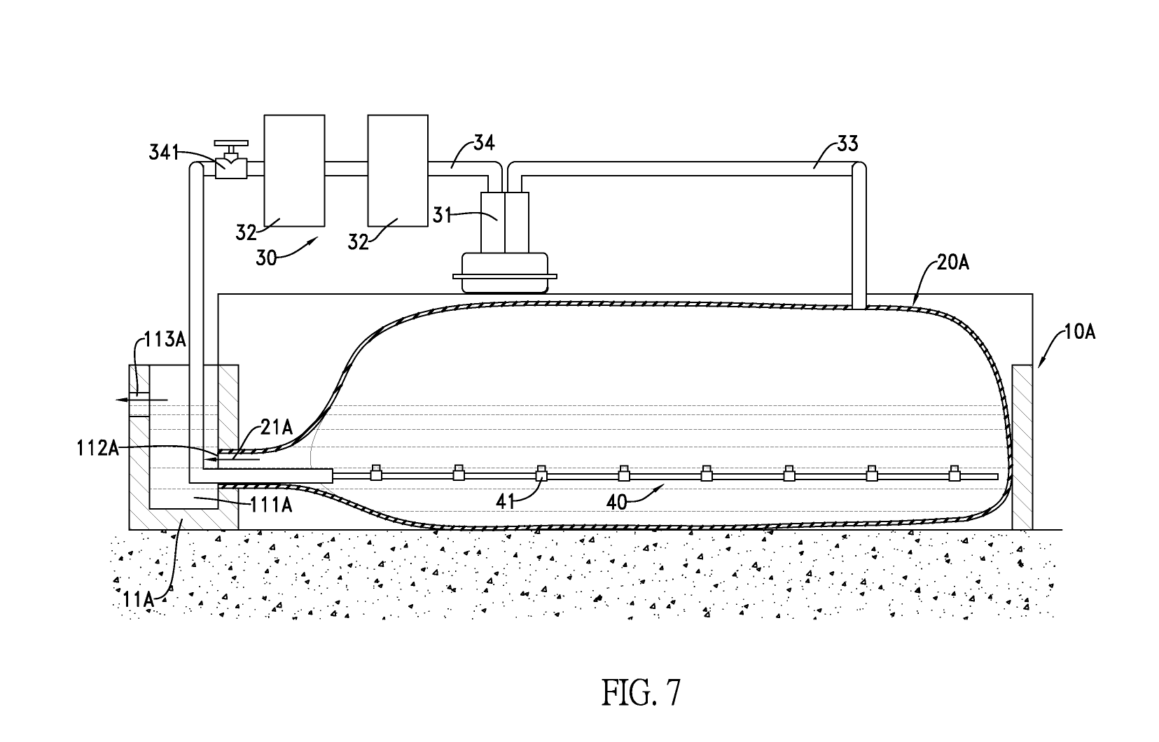

With reference to FIGS. 5 to 7, when the second embodiment of the present invention is in use, the organic substances 50 and culture fluid of the anaerobic bacteria are fed into the container 20A via the gateway 111A of the transporting base 11A and the opening 21A and bury the spray pipe 40. The container 20A is sealed by the organic substances 50, so the container 20, 20B is isolated from the air. Then, the anaerobic bacteria start reproducing themselves in the container 20, 20B and fermenting the organic substances 50. The organic substances 50 are decomposed to generate biogas, biogas slurry, and organic manure. The biogas floats at the top side in the container 20A due to the light weight property. The biogas slurry and the organic manure are located at the bottom side of the container 20, 20B. After the biogas is generated, the pump 31 of the circulation unit 30 will start to draw biogas to store in the storage tanks 32 via the pumping pipe 33. And the gas valve 341 is switched on and makes the conveying pipe 34 connected. So the biogas can be flowed into the spray pipe 40. The biogas is spewed out of the spray pipe 40 at short intervals of time via each one of the jets 41 to stir and mix the organic substances 50. After the anaerobic fermentation of the organic substances 50 is completed, all of the biogas will be stored in the multiple storage tanks 32. The biogas fluid is discharged from the outlet 113A via the gateway 111A of the transporting base 11A.

According to the aforementioned technique features, the present invention can convey the biogas generated from the anaerobic fermentation into the spray pipe 40, and the biogas is spewed via the multiple jets at short intervals of time to stir and mix the organic substances 50 for promoting the rate of anaerobic fermentation and improving the ecological distribution of the anaerobic bacteria.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An anaerobic fermentation with venturi stirring equipment comprising:
a container having at least one opening;
a circulation unit communicating with the container, and having
a pump disposed out of the container;
a pumping pipe communicating with the pump and a top of the container; and
a conveying pipe communicating with the pump, mounted through one of the at least one opening of the container, and having
two ends, one of the two ends of the conveying pipe communicating with the pump;
a gas valve mounted on the conveying pipe;
at least one storage tank mounted on the conveying pipe; and
a spray pipe mounted in the container and communicating with the other one of the two ends of the conveying pipe.

2. The anaerobic fermentation with venturi stirring equipment as claimed in claim 1, wherein
the container is made of cement or bricks and has
a surrounding wall having a top opening;
a cover hermetically mounted on the top opening of the surrounding wall; and
an inner space formed between the surrounding wall and the cover, and communicating with the at least one opening of the container;
the at least one opening of the container includes
a first opening formed through the surrounding wall of the container; and
a second opening formed through the surrounding wall of the container;
the pumping pipe of the circulation unit is connected to the cover;
the conveying pipe of the circulation unit is located in the inner space of the container.

3. The anaerobic fermentation with venturi stirring equipment as claimed in claim 1 further comprising an enclosure having
a surrounding wall;
a space surrounded by the surrounding wall;
a first transporting base formed on the surrounding wall and having
a first gateway formed in the first transporting base; and
a first connecting opening formed through the surrounding wall and communicating with the first gateway and the space;
a second transporting base mounted on the surrounding wall opposite to the first transporting base and having
a second gateway formed in the second transporting base; and
a second connecting opening formed through the surrounding wall and communicating with the second gateway and the space.

4. The anaerobic fermentation with venturi stirring equipment as claimed in claim 3, wherein
the container is a bag, is mounted in the space of the enclosure, and has
a first opening formed through the container, facing the first transporting base, and communicating with the first gateway of the first transporting base via the first connecting opening; and
a second opening formed through the container, facing the second transporting base, and communicating with the second gateway of the second transporting base via the second connecting opening.

5. The anaerobic fermentation with venturi stirring equipment as claimed in claim 1, wherein the spray pipe has multiple jets mounted upward on the spray pipe, and the multiple jets are spaced from each other at intervals.

6. The anaerobic fermentation with venturi stirring equipment as claimed in claim 4, wherein the spray pipe has multiple jets mounted upward on the spray pipe, and the multiple jets are spaced from each other at intervals.

7. The anaerobic fermentation with venturi stirring equipment as claimed in claim 3, wherein the second transporting base has an outlet formed through the second transporting base and communicating with the second gateway.

8. The anaerobic fermentation with venturi stirring equipment as claimed in claim 6, wherein the second transporting base has an outlet formed through the second transporting base and communicating with the second gateway.

9. The anaerobic fermentation with venturi stirring equipment as claimed in claim 1 further comprising an enclosure having
a surrounding wall;
a space surrounded by the surrounding wall; and
a transporting base mounted on the surrounding wall and having
a gateway formed in the transporting base;
a connecting opening formed through the surrounding wall and communicating with the gateway and the space; and
an outlet formed through the transporting base and communicating with the gateway.

10. The anaerobic fermentation with venturi stirring equipment as claimed in claim 9, wherein the container is a bag, is mounted in the space of the enclosure, and has
an opening formed through the container, facing the transporting base of the enclosure, and communicating with the gateway of the transporting base via the connecting opening.

11. The anaerobic fermentation with venturi stirring equipment as claimed in claim 3, wherein the circulation unit has multiple storage tanks disposed out of the enclosure.
